(19) United States
(12) Patent Application Publication
(10) Pub. No.: US 2025/0352048 A1
(43) Pub. Date: Nov. 20, 2025

(54) MEDICAL DEVICE, ENDOSCOPE SYSTEM, CONTROL METHOD, AND COMPUTER-READABLE RECORDING MEDIUM

(71) Applicant: OLYMPUS MEDICAL SYSTEMS CORP., Tokyo (JP)

(72) Inventors: Yasuo TANIGAMI, Tokyo (JP); Yusuke OTSUKA, Yokohama-shi (JP); Noriko KURODA, Tokyo (JP); Takaaki IGARASHI, Tokyo (JP)

(73) Assignee: OLYMPUS MEDICAL SYSTEMS CORP., Tokyo (JP)

(21) Appl. No.: 19/287,964

(22) Filed: Aug. 1, 2025

Related U.S. Application Data
(63) Continuation of application No. PCT/JP2023/004407, filed on Feb. 9, 2023.

(51) Int. Cl. A61B 1/04; A61B 1/00; A61B 1/045; A61B 1/05

(52) U.S. Cl. CPC A61B 1/043; A61B 1/00009; A61B 1/0005; A61B 1/045; A61B 1/05

(57) ABSTRACT

A medical device includes a processor configured to: obtain a tissue image, obtain a fluorescence image, identify a correspondence relationship between the tissue image and the fluorescence image, obtain relationship information on a correlation between a fluorescence intensity in the fluorescence image and a degree of thermal invasiveness, identify an insufficient heat denaturation region, identify an excess heat denaturation region, identify an appropriate heat denaturation region, add heat denaturation information to the tissue image based on the correspondence relationship, superimpose the insufficient heat denaturation region, the excess heat denaturation region, and the appropriate heat denaturation region on the tissue image, and display resultant image in a display.

### CROSS REFERENCES TO RELATED APPLICATIONS

This application is a continuation of International Application No. PCT/JP2023/004407, filed on Feb. 9, 2023, the entire contents of which are incorporated herein by reference.

### BACKGROUND

### 1. Technical Field

The present disclosure relates to a medical device, an endoscope system, a control method, and a computer-readable recording medium.

### 2. Related Art

In the related art, a technology is known that, at the time of performing heat treatment on the body tissue using an energy device, enables visualization of the state of heat denaturation of the body tissue (for example, refer to International Laid-open Pamphlet No. 2020/054723).

In the technology disclosed in International Laid-open Pamphlet No. 2020/054723, based on a taken image that captures the fluorescence generated from the body tissue when bombarded with an excitation light, the state of heat denaturation of the body tissue is visualized. More particularly, in the technology disclosed in International Laid-open Pamphlet No. 2020/054723, from among all of the pixels of the taken image, the regions in which the fluorescence intensity is higher than the preset fluorescence intensity are displayed as the regions having high heat denaturation.

### SUMMARY

In some embodiments, a medical device includes a processor configured to: obtain a tissue image including a target for a heat treatment, obtain a fluorescence image that is taken by an imaging sensor, identify a correspondence relationship between the tissue image and the fluorescence image, obtain relationship information on a correlation between a fluorescence intensity in the fluorescence image and a degree of thermal invasiveness, identify an insufficient heat denaturation region based on the fluorescence image and the relationship information, identify an excess heat denaturation region based on the fluorescence image and the relationship information, identify an appropriate heat denaturation region based on the fluorescence image and the relationship information, add heat denaturation information including the insufficient heat denaturation region, the excess heat denaturation region, and the appropriate heat denaturation region, to the tissue image based on the correspondence relationship between the tissue image and fluorescence image, and superimpose the insufficient heat denaturation region, the excess heat denaturation region, and the appropriate heat denaturation region on the tissue image such that the insufficient heat denaturation region, the excess heat denaturation region, and the appropriate heat denaturation region are identifiable from each other, and display resultant image in a display.

In some embodiments, an endoscope system includes: a light source configured to emit an excitation light; an endoscope configured to output a taken image which is taken by an imaging sensor; and a medical device including a processor configured to process the taken image, the processor being configured to obtain a tissue image including a target for a heat treatment, obtain a fluorescence image that is taken by the imaging sensor, identify a correspondence relationship between the tissue image and the fluorescence image, obtain relationship information on a correlation between a fluorescence intensity in the fluorescence image and a degree of thermal invasiveness, identify an insufficient heat denaturation region based on the fluorescence image and the relationship information, identify an excess heat denaturation region based on the fluorescence image and the relationship information, identify an appropriate heat denaturation region based on the fluorescence image and the relationship information, add heat denaturation information including the insufficient heat denaturation region, the excess heat denaturation region, and the appropriate heat denaturation region, to the tissue image based on the correspondence relationship between the tissue image and fluorescence image, and superimpose the insufficient heat denaturation region, the excess heat denaturation region, and the appropriate heat denaturation region on the tissue image such that the insufficient heat denaturation region, the excess heat denaturation region, and the appropriate heat denaturation region are identifiable from each other, and display resultant image in a display.

In some embodiments, provided is a control method implemented in a medical device. The method includes: obtaining a tissue image including a target for a heat treatment, obtaining a fluorescence image that is taken by an imaging sensor, identifying a correspondence relationship between the tissue image and the fluorescence image, obtaining relationship information on a correlation between a fluorescence intensity in the fluorescence image and a degree of thermal invasiveness, identifying an insufficient heat denaturation region based on the fluorescence image and the relationship information, identifying an excess heat denaturation region based on the fluorescence image and the relationship information, identifying an appropriate heat denaturation region based on the fluorescence image and the relationship information, adding heat denaturation information including the insufficient heat denaturation region, the excess heat denaturation region, and the appropriate heat denaturation region, to the tissue image based on the correspondence relationship between the tissue image and fluorescence image, and superimposing the insufficient heat denaturation region, the excess heat denaturation region, and the appropriate heat denaturation region on the tissue image such that the insufficient heat denaturation region, the excess heat denaturation region, and the appropriate heat denaturation region are identifiable from each other, and displaying resultant image in a display.

In some embodiments, provided is a non-transitory computer-readable recording medium with an executable program stored thereon. The program cause a medical device to execute: obtaining a tissue image including a target for a heat treatment, obtaining a fluorescence image that is taken by an imaging sensor, identifying a correspondence relationship between the tissue image and the fluorescence image, obtaining relationship information on a correlation between a fluorescence intensity in the fluorescence image and a degree of thermal invasiveness, identifying an insufficient heat denaturation region based on the fluorescence image and the relationship information, identifying an excess heat denaturation region based on the fluorescence image and the relationship information, identifying an appropriate heat denaturation region based on the fluorescence image and the relationship information, adding heat denaturation information including the insufficient heat denaturation region, the excess heat denaturation region, and the appropriate heat denaturation region, to the tissue image based on the correspondence relationship between the tissue image and fluorescence image, and superimposing the insufficient heat denaturation region, the excess heat denaturation region, and the appropriate heat denaturation region on the tissue image such that the insufficient heat denaturation region, the excess heat denaturation region, and the appropriate heat denaturation region are identifiable from each other, and displaying resultant image in a display.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of an endoscope system according to an embodiment;

FIG. 2 is a block diagram illustrating a functional configuration of the main parts of the endoscope system according to the present embodiment;

FIG. 3 is a diagram illustrating the wavelength characteristics of an excitation light emitted by a second light source;

FIG. 4 is a diagram illustrating the transmission characteristics of a cut filter;

FIG. 5 is a diagram for explaining the principle of observation applied in a fluorescence observation mode;

FIG. 6 is a diagram for explaining the principle of observation applied in a normal light observation mode;

FIG. 7 is a flowchart for explaining a pre-heat-treatment control method;

FIGS. 8 and 9 are diagrams for explaining the pre-heat-treatment control method;

FIG. 10 is a flowchart for explaining a control method performed when heat treatment is carried out; and

FIGS. 11 to 15 are diagrams for explaining the control method performed when heat treatment is carried out.

### DETAILED DESCRIPTION

An illustrative embodiment (hereinafter, called an embodiment) is disclosed below with reference to the accompanying drawings. However, the disclosure is not limited by the embodiment described below. Moreover, in the drawings, identical constituent elements are referred to by the same reference numerals.

### Overall Configuration of Endoscope System

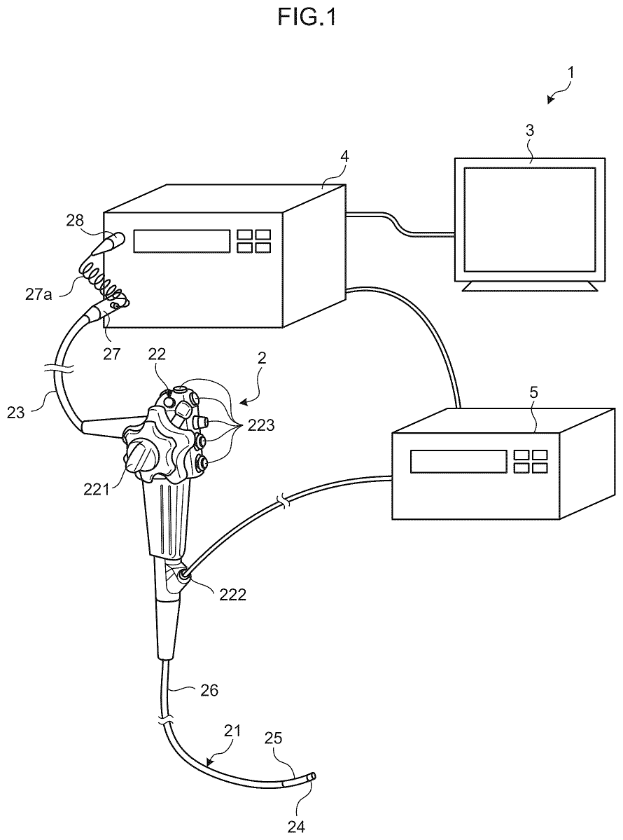

FIG. 1 is a diagram illustrating an overall configuration of an endoscope system 1 according to the embodiment.

The endoscope system 1 according to the embodiment is used in peroral endoscopic myotomy (POEM). More particularly, in peroral endoscopic myotomy, an insertion portion 21 of an endoscope 2 is inserted from the mouth into the esophagus of the subject and an in-vivo image of the subject is taken; and a display image based on the obtained image data is displayed in a display device 3. Then, while checking the display image, the operator makes an incision in (performs heat treatment on) the excessively-developed muscles of the esophagus and the cardia.

As illustrated in FIG. 1, the endoscope system 1 includes the endoscope 2, the display device 3, a control device 4, and a heat treatment device 5.

The endoscope 2 generates image data (RAW data) of in-vivo images of the subject, and outputs the image data to the control device 4. As illustrated in FIG. 1, the endoscope 2 includes the insertion portion 21, an operating unit 22, and a universal cord 23.

The insertion portion 21 is at least partially flexible and is inserted inside the subject. As illustrated in FIG. 1, the insertion portion 21 includes a front end portion 24 at the front end thereof; a freely-bendable curved portion 25 that is connected to the proximal end of the front end portion 24 (i.e., the side toward the operating unit 22); and a flexible tube 26 that is a flexible and long tube connected to the proximal end of the curved portion 25.

The operating unit 22 is connected to the proximal end portion of the insertion portion 21. The operating unit 22 receives various operations performed with respect to the endoscope 2. As illustrated in FIG. 1, the operating unit 22 includes a bending knob 221, an insertion opening 222, and a plurality of operating members 223.

The bending knob 221 is configured to be rotationally movable according to a user operation performed by the user such as an operator. As a result of the rotational movement of the bending knob 221, a bending mechanism (not illustrated) that is made of a metal wire or a resin wire and that is disposed inside the insertion portion 21 is operated. With that, the curved portion 25 bends.

The insertion opening 222 is communicated with a treatment tool channel (not illustrated) that is a pipe conduit extending from the front end of the insertion portion 21, and serves as the insertion opening for inserting a treatment tool from the outside of the endoscope 2 into the treatment tool channel.

The operating members 223 are configured using buttons for receiving various operations performed by the user such as an operator; and output operation signals corresponding to the various operations to the control device 4 via the universal cord 23. Examples of the various operations include an operation for switching the observation mode of the endoscope system 1 among a normal-light observation mode, a fluorescence observation mode, and a specific observation mode.

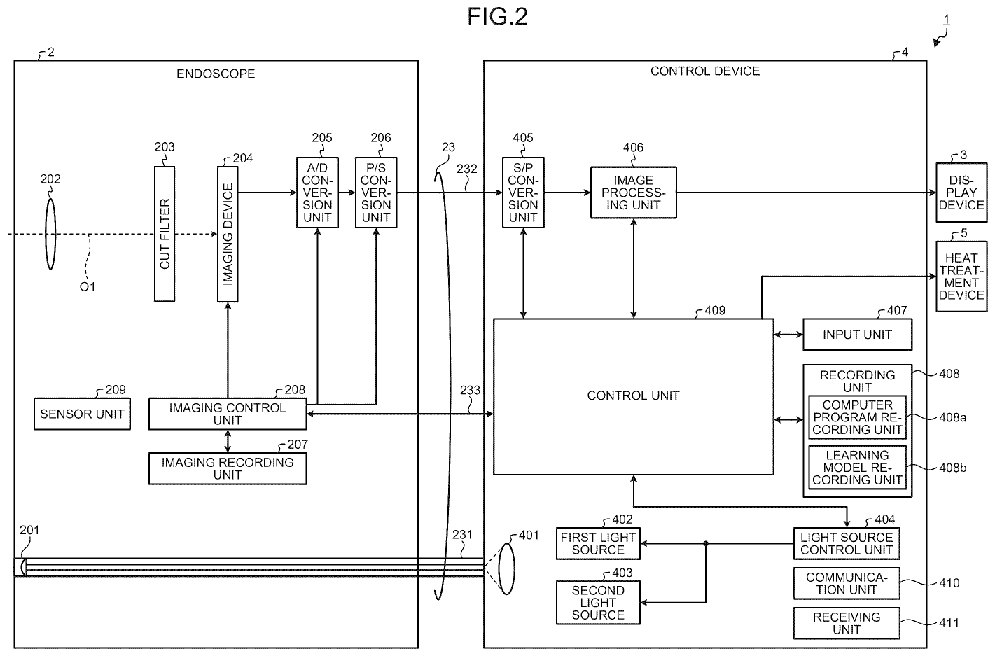

The universal cord 23 extends from the operating unit 22 in a different direction than the direction of extension of the insertion portion 21; and has a light guide 231 (see FIG. 2) made of an optical fiber arranged thereon, has a first signal line 232 (see FIG. 2) arranged thereon for transmitting the image data, and has a second signal line 233 (see FIG. 2) arranged thereon for transmitting the operation signals. Moreover, as illustrated in FIG. 1, a first connector portion 27, a second connector portion 28, and a cable 27a are disposed at the proximal end of the universal cord 23.

The first connector portion 27 is connected to the control device 4 in a detachably attachable manner.

The cable 27a is a coiled cable extending from the first connector portion 27.

The second connector portion 28 is provided at the front end of the cable 27a and is connected to the control device 4 in a detachably attachable manner.

The display device 3 is configured using a display monitor such as a liquid crystal display or an organic electroluminescence (EL) display; and, under the control performed by the control device 4, displays a display image based on the image data having been subjected to image processing in the control device 4 and displays a variety of information related to the endoscope system 1.

The control device 4 is equivalent to a medical device. The control device 4 is implemented using a processor representing a processing device equipped with hardware such as a graphics processing unit (GPU), a field programmable gate array (FPGA), or a central processing unit (CPU); and using a memory representing a temporary memory area used by the processor. According to the computer programs stored in the memory, the control device 4 comprehensively controls the operations of the constituent elements of the endoscope system 1.

The heat treatment device 5 is, for example, an energy device such as a high-frequency knife that performs heat treatment on the body tissue by supplying a high-frequency current to the body tissue, or a laser irradiation device that performs heat treatment on the body tissue by irradiating the body tissue with a high-output infrared laser. More particularly, the heat treatment device 5 is inserted from the insertion opening 222 into the esophagus via the treatment tool channel provided inside the insertion portion 21. Then, according to a user operation performed by the user such as an operator, the heat treatment device 5 performs heat treatment on the muscles of the esophagus and the cardia.

### Functional Configuration of Main Parts of Endoscope System

Given below is the explanation of a functional configuration of the main parts of the endoscope system 1.

FIG. 2 is a block diagram illustrating a functional configuration of the main parts of the endoscope system 1.

The following explanation is given about the endoscope 2 and the control device 4 in that order.

### Configuration of Endoscope

Firstly, the explanation is given about a configuration of the endoscope 2.

As illustrated in FIG. 2, the endoscope 2 includes an illumination optical system 201, an imaging optical system 202, a cut filter 203, an imaging device 204, an A/D conversion unit 205, a P/S conversion unit 206, an imaging recording unit 207, an imaging control unit 208, and a sensor unit 209.

The illumination optical system 201, the imaging optical system 202, the cut filter 203, the imaging device 204, the A/D conversion unit 205, the P/S conversion unit 206, the imaging recording unit 207, the imaging control unit 208, and the sensor unit 209 are disposed in the front end portion 24.

The illumination optical system 201 is configured using one or more lenses; and bombards an illumination light, which is supplied from the light guide 231, toward the subject.

The imaging optical system 202 is configured using one or more lenses; and condenses the lights such as the reflected light that has reflected from the subject, the optical feedback coming from the subject, and the fluorescence emitted by the subject, and forms a subject image on the light receiving surface of the imaging device 204.

The cut filter 203 is disposed on an optical axis L1 of the imaging optical system 202 and in between the imaging optical system 202 and the imaging device 204. The cut filter 203 blocks the lights having predetermined wavelength bands and allows passage of other lights.

Regarding the transmission characteristics of the cut filter 203, the explanation is given later in the section “configuration of control device”.

The imaging device 204 is configured using an image sensor in which any one of the color filters constituting a Bayer layout (RGGB) is disposed in each of a plurality of pixels arranged in a two-dimensional matrix and such image sensor includes an image sensor of a charge coupled device (CCD) or a CMOS (CMOS stands for Complementary Metal Oxide Semiconductor). Then, under the control performed by the imaging control unit 208, the imaging device 204 receives light of the subject image that is formed by the imaging optical system 202 and that has passed through the cut filter 203; performs photoelectric conversion; and generates a taken image (an analog signal). In the present embodiment, the imaging device 204 is configured, in an integrated manner, with a TOF sensor (TOF stands for Time Of Flight) that obtains subject distance information (hereinafter, called depth map information) according to the TOF method. The depth map information indicates the information in which, for each pixel position in the taken image, the subject distance is detected from the position of the imaging device 204 (the position of the front end portion 24) to the corresponding position on the observation target corresponding to the concerned pixel position.

Meanwhile, the configuration for generating the depth map information is not limited to using the TOF sensor, and it is alternatively possible to use a phase difference sensor or a stereo camera.

In the following explanation, the depth map information and the taken image are collectively referred to as the image data.

Subsequently, the imaging device 204 outputs the image data to the A/D conversion unit 205.

The A/D conversion unit 205 is configured using an A/D conversion circuit and, under the control performed by the imaging control unit 208, performs A/D conversion with respect to the analog image data input from the imaging device 204; and outputs the post-conversion image data to the P/S conversion unit 206.

The P/S conversion unit 206 is configured using a P/S conversion circuit and, under the control of the imaging control unit 208, performs parallel/serial conversion with respect to the digital image data input from the A/D conversion unit 205; and outputs the post-conversion image data to the control device 4 via the first signal line 232.

Meanwhile, instead of using the P/S conversion unit 206, it is possible to use an E/O conversion unit that converts the image data into optical signals, so that the image data in the form of optical signals is sent to the control device 4. Moreover, for example, the image data can be sent to the control device 4 using wireless communication such as Wi-Fi (Wireless Fidelity) (registered trademark).

The imaging recording unit 207 is configured using a nonvolatile memory or a volatile memory, and is used to record a variety of information related to the endoscope 2 (for example, the pixel information of the imaging device 204 and the characteristics of the cut filter 203). Moreover, the imaging recording unit 207 is used to record a variety of setting data and control parameters that are sent from the control device 4 via the second signal line 233.

The imaging control unit 208 is implemented using a timing generator (TG), a processor that represents a processing device equipped with hardware such as a CPU, and a memory that represents a temporary memory area used by the processor. Based on the setting data received from the control device 4 via the second signal line 233, the imaging control unit 208 controls the constituent elements such as the imaging device 204, the A/D conversion unit 205, and the P/S conversion unit 206.

The sensor unit 209 is a sensor used in calculating the position of the front end of the insertion portion 21 (i.e., the position of the front end portion 24) and calculating the direction in which the front end of the insertion portion 21 is oriented (i.e., the field of view of the front end). In the present embodiment, the sensor unit 209 is configured using a plurality of magnetic coils that generate magnetism.

### Configuration of Control Device

Given below is the explanation of a configuration of the control device 4.

As illustrated in FIG. 2, the control device 4 includes a condenser lens 401, a first light source 402, a second light source 403, a light source control unit 404, an S/P conversion unit 405, an image processing unit 406, an input unit 407, a recording unit 408, a control unit 409, a communication unit 410, and a receiving unit 411.

The condenser lens 401 condenses the lights emitted by the first light source 402 and the second light source 403, and emits the condensed light to the light guide 231.

Under the control performed by the light source control unit 404, the first light source 402 emits the white light (normal light) representing the visible light, and supplies the white light as the illumination light to the light guide 231. The first light source 402 is configured using a collimating lens, a white LED lamp (LED stands for Light Emitting Diode), and a driver.

Alternatively, as the first light source 402; a red LED lamp, a green LED lamp, and a blue LED lamp can be made to emit the lights in a simultaneous manner, so that the white light can be provided as the visible light. Still alternatively, the first light source 402 can be configured using a halogen lamp or a xenon lamp.

Under the control performed by the light source control unit 404, the second light source 403 emits an excitation light having a predetermined wavelength band, and supplies the excitation light as the illumination light to the light guide 231.

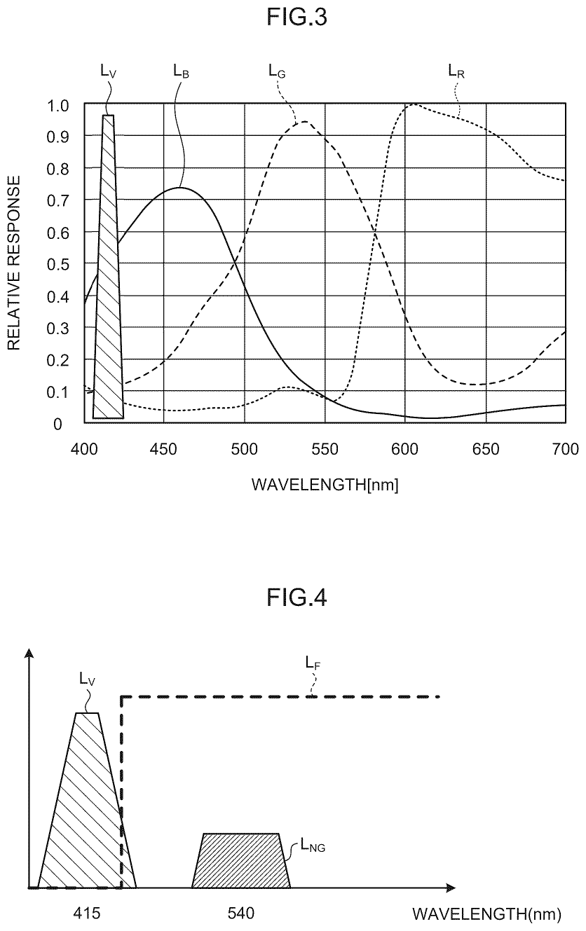

FIG. 3 is a diagram illustrating the wavelength characteristics of the excitation light emitted by the second light source 403. More particularly, in FIG. 3, the horizontal axis represents the wavelength (nm), and the vertical axis represents the wavelength characteristics. Moreover, in FIG. 3, a curved line Ly represents the wavelength characteristics of the excitation light emitted by the second light source 403. Moreover, in FIG. 3, a curved line L_{B} represents the wavelength characteristics of the blue color, a curved line LG represents the wavelength characteristics of the green color, and a curved line LR represents the wavelength characteristics of the red color.

In the present embodiment, as illustrated in FIG. 3, the second light source 403 has the central wavelength (peak wavelength) of 415 nm, and emits an excitation light having the wavelength band between 400 nm and 430 nm. The second light source 403 is configured using a collimating lens, a semiconductor laser such as a violet laser diode (LD), and a driver.

Given below is the explanation of the transmission characteristics of the cut filter 203.

FIG. 4 is a diagram illustrating the transmission characteristics of the cut filter 203. More particularly, in FIG. 4, the horizontal axis represents the wavelength (nm) and the vertical axis represents the wavelength characteristics. Moreover, in FIG. 4, a curved line L_{F} represents the transmission characteristics of the cut filter 203, and a curved line L_{V} represents the wavelength characteristics of the excitation light. Furthermore, in FIG. 4, a curved line L_{NG} represents the wavelength characteristics of the fluorescence generated as a result of bombarding an excitation light on an advanced glycation end product that is generated due to the heat treatment performed on the body tissue.

In the present embodiment, as illustrated in FIG. 4, the cut filter 203 blocks some of the excitation light that is reflected from the body tissue in the observation region, and allows passage of the light having other wavelength bands including the fluorescence component. More particularly, the cut filter 203 blocks some of the light that has the wavelength bands of small wavelengths between 400 nm and smaller than 430 nm and that includes the excitation light, and allows passage of the light that has the wavelength bands of longer wavelengths than 430 nm and that includes the fluorescence generated as a result of bombarding an excitation light on an advanced glycation end product generated due to the heat treatment performed on the body tissue.

The light source control unit 404 is implemented using a processor representing a processing device equipped with hardware such as an FPGA or a CPU, and using a memory representing a temporary memory area used by the processor. Then, based on the control data input from the control unit 409, the light source control unit 404 controls the light emission timings and the light emission periods for the first light source 402 and the second light source 403. Under the control performed by the control unit 409, the S/P conversion unit 405 performs serial/parallel conversion of the image data received from the endoscope 2 via the first signal line 232, and outputs the post-conversion image data to the image processing unit 406.

When the endoscope 2 outputs the image data using optical signals, the S/P conversion unit 405 can be substituted with an O/E conversion unit that converts the optical signals into electrical signals. Moreover, when the endoscope 2 sends the image data using wireless communication, the S/P conversion unit 405 can be substituted with a communication module capable of receiving wireless signals.

The image processing unit 406 is equivalent to a processor. The image processing unit 406 is implemented using a processor representing a processing device equipped with hardware such as a GPU or an FPGA, and using a memory representing a temporary memory area used by the processor. Under the control performed by the control unit 409, the image processing unit 406 performs predetermined image processing on the image data input as the parallel data from the S/P conversion unit 405, and outputs the resultant image data to the display device 3. Examples of the predetermined image processing include color reconstruction, white balancing, gain adjustment, Y correction, and format conversion.

The input unit 407 is configured using a mouse, or a footswitch, or a keyboard, or buttons, or switches, or a touch-sensitive panel. The input unit 407 receives a user operation performed by the user such as an operator, and outputs an operation signal corresponding to the user operation to the control unit 409.

The recording unit 408 is configured using a volatile memory, or a nonvolatile memory, or a solid state drive (SSD), or a hard disk drive (HDD), or a recording medium such as a memory card. The recording unit 408 is used to record various parameters required for the operations performed in the endoscope system 1. The recording unit 408 includes a computer program recording unit 408a for recording various computer programs written to enable operations in the endoscope system 1, and a learning model recording unit 408b explained below.

The learning model recording unit 408b is used to record a learning model that is used by the control unit 409 while performing image recognition. For example, the learning model is generated as a result of performing machine learning using artificial intelligence (AI).

More particularly, the learning model is obtained when the image data obtained by capturing the treatment target range for performing heat treatment during peroral endoscopic myotomy (POEM) is treated as the teacher data, and when machine learning (for example, deep learning) of the treatment target range is performed based on the teacher data.

The control unit 409 is equivalent to a processor. The control unit 409 is implemented using a processor representing a processing device equipped with hardware such as an FPGA or a CPU, and using a memory representing a temporary memory area used by the processor. The control unit 409 comprehensively controls the constituent elements of the endoscope system 1.

The communication unit 410 is an interface that communicates a variety of data with an external tomographic device (not illustrated), such as a CT device (CT stands for Computed Tomography) or an MRI device (MRI stands for Magnetic Resonance Imaging), according to a predetermined protocol. Then, under the control performed by the control unit 409, the communication unit 410 obtains a tissue image representing a tomographic image taken by the tomographic device.

The communication between the communication unit 410 and an external tomographic device can be performed either using wireless communication or using wired communication. Alternatively, the configuration can be such that a tomographic image (tissue image) taken by a tomographic device is stored in a server, and the communication unit 410 obtains the tomographic image (tissue image) from the server.

The receiving unit 411 receives the magnetism coming from the sensor unit 209. Then, the receiving unit 411 outputs, to the control unit 409, a signal corresponding to the received magnetism.

### Principle of Observation Applied in Observation Modes of Endoscope System

Given below is the explanation about the principle of observation applied in the observation modes of the endoscope system 1.

The following explanation is given about the fluorescence observation mode and the normal light observation mode in that order.

### Principle of Observation Applied in Fluorescence Observation Mode

Firstly, the explanation is given about the principle of observation applied in the fluorescence observation mode.

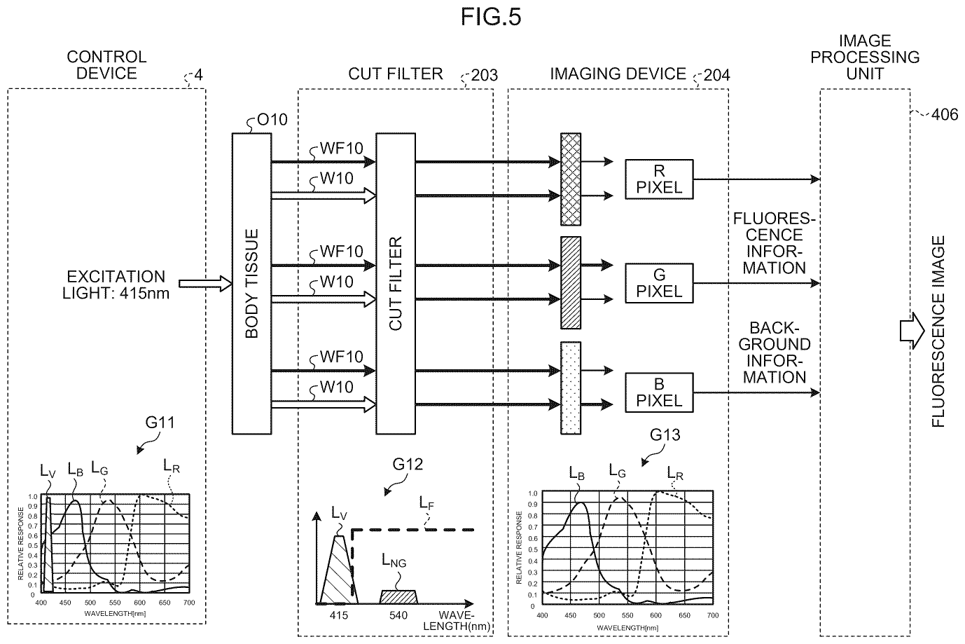

FIG. 5 is a diagram for explaining the principle of observation applied in the fluorescence observation mode.

As illustrated in a graph G11 in FIG. 5, firstly, the control device 4 causes the second light source 403 to emit a light, so that an excitation light (having the central wavelength of 415 nm) is bombarded on a body tissue O10. In that case, as illustrated in a graph G12 in FIG. 5, the reflected light (hereinafter, referred to as a reflected light W10), which at least includes the component of the excitation light reflected from the body tissue O10 and includes the return light, gets blocked by the cut filter 203 and undergoes a decrease in the intensity. On the other hand, some of the components having longer wavelengths than the wavelength band of the blocked light fall on the imaging device 204 without getting blocked.

More particularly, as illustrated in the graph G12 in FIG. 5, the cut filter 203 blocks the major portion of the reflected light W10, which falls on the G pixels in the imaging device 204 and which has the wavelength bands of small wavelengths including the wavelength band of the excitation light; and allows passage of the wavelength bands of longer wavelengths than the blocked wavelength bands. Furthermore, as illustrated in the graph G12 in FIG. 5, the cut filter 203 allows passage of a fluorescence WF10 that is the autofluorescence of the advanced glycation end product generated as a result of performing heat treatment on the body tissue O10. For that reason, the reflected light W10 having a decreased intensity and the fluorescence WF10 falls on each of the R pixels, the G pixels, and the B pixels of the imaging device 204.

The G pixels of the imaging device 204 have sensitivity to the fluorescence WF10. However, as illustrated by the curved line L_{NG} having the fluorescence characteristics as illustrated in the graph G12 in FIG. 5, the fluorescence is only a minor reaction. For that reason, the output value corresponding to the fluorescence WF10 in the G pixels represents only a small value.

Then, the image processing unit 406 obtains the image data (RAW data) from the imaging device 204; performs image processing with respect to the output value of each G pixel and each B pixel included in the image data; and generates a fluorescence image. In that case, the output values of the G pixels include fluorescence information corresponding to the fluorescence WF10, which is generated from the heat treatment region (advanced glycation end product) used for performing heat treatment on the body tissue O10. The output values of the B pixels include background information obtained from the body tissue O10 of the subject in which the heat treatment region is included. When that fluorescence image is displayed in the display device 3, it becomes possible to observe the heat treatment region used for performing heat treatment on the body tissue O10.

### Principle of Observation Applied in Normal Light Observation Mode

Given below is the explanation about the principle of observation applied in the normal light observation mode.

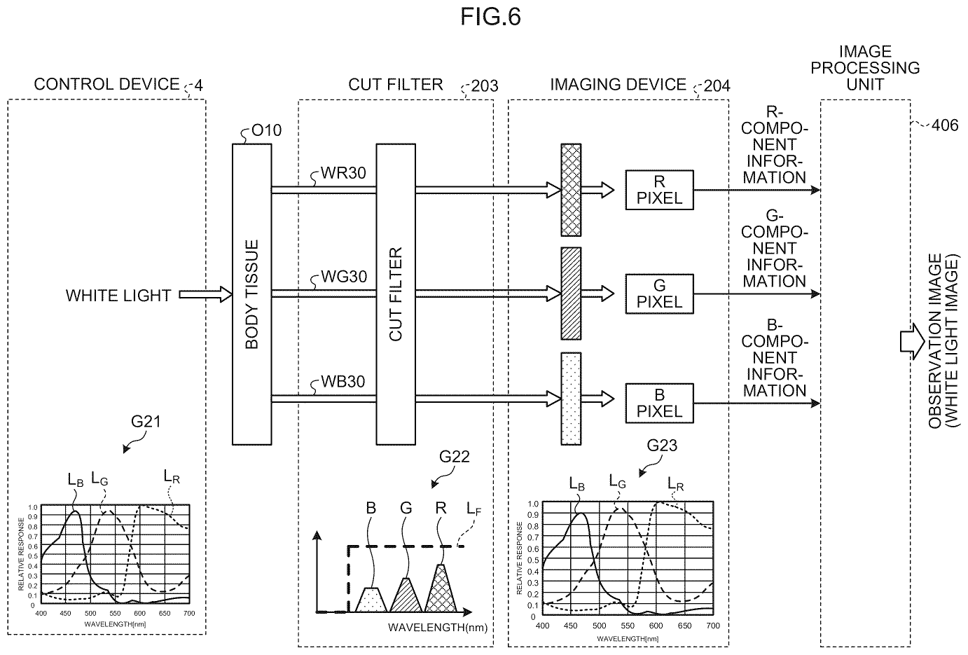

FIG. 6 is a diagram for explaining the principle of observation applied in the normal light observation mode.

As illustrated in a graph G21 in FIG. 6, firstly, the control device 4 causes the first light source 402 to emit a light, so that the white light is bombarded on the body tissue O10. In that case, of the reflected lights reflected from the body tissue O10 (hereinafter, written as reflected lights WR30, WG30, and WB30) and the return light, some light is blocked by the cut filter 203, and the remaining light falls on the imaging device 204. More particularly, as illustrated in a graph G22 in FIG. 6, the cut filter 203 blocks the reflected light having the wavelength bands of small wavelengths including the wavelength band of the excitation light. Hence, the component of the light having the wavelength band of the blue color, which falls on the B pixels of the imaging device 204, becomes smaller as compared to the state in which the cut filter 203 is not disposed.

Then, the image processing unit 406 obtains the image data (RAW data) from the imaging device 204; performs image processing with respect to the output value of each R pixel, each G pixel, and each B pixel included in the image data; and generates an observation image (white light image). In that case, since the blue color component included in the image data is smaller as compared to the state in which the cut filter 203 is not disposed, the image processing unit 406 performs white balancing for adjusting the white balance in order to ensure that the ratio of the red color component, the green color component, and the blue color component becomes constant. Thus, when the observation image (white light image) is displayed in the display device 3, it becomes possible to observe a natural observation image (white light image) regardless of whether the cut filter 203 is disposed.

### Control Method

Given below is the explanation of a control method implemented by the control device 4.

The following explanation is given about a control method implemented by the control device 4 before the heat treatment device 5 performs heat treatment on the body tissue (hereinafter, called a pre-heat-treatment control method), and about a control method implemented by the control device 4 at the time when the heat treatment is performed (hereinafter, called a control method performed when heat treatment is carried out).

### Pre-Heat-Treatment Control Method

Firstly, the explanation is given about the control method implemented by the control device 4 before the heat treatment device 5 performs heat treatment on the body tissue.

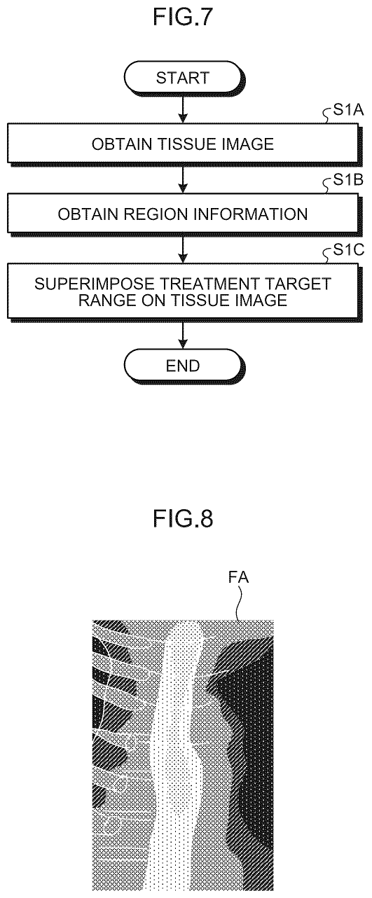

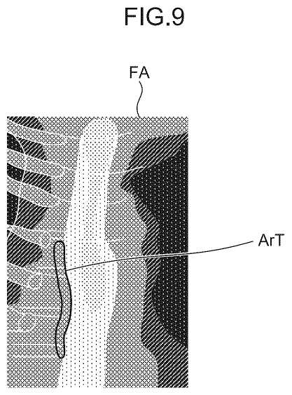

FIG. 7 is a flowchart for explaining the pre-heat-treatment control method. FIGS. 8 and 9 are diagrams for explaining the pre-heat-treatment control method. More particularly, FIG. 8 is a diagram illustrating a tissue image FA obtained at Step S1A. FIG. 9 is a diagram that corresponds to FIG. 8 and that explains the operation performed at Step S1C.

According to a user operation performed using the input unit 407, the control unit 409 controls the operation of the communication unit 410, and obtains the tissue image FA (see FIG. 8) from an external tomographic device via the communication unit 410 (Step S1A).

The tissue image FA is a tomographic image obtained by capturing the same subject as the target subject for which the heat treatment is performed by the heat treatment device 5, by an external tomographic device. In the tissue image FA, for each pixel, three-dimensional coordinates of the corresponding position in the observation target that corresponds to the concerned pixel position are added to the concerned pixel. The three-dimensional coordinates are based on a specific coordinate system and are calculated by the tomographic device.

After Step S1A, the control unit 409 obtains range information indicating the treatment target range for performing heat treatment in the tissue image FA (Step S1B).

More particularly, at Step S1B, the control unit 409 performs image recognition using the learning model recorded in the learning model recording unit 408b, and obtains (extracts) the range information indicating a treatment target range ArT (see FIG. 9) for performing heat treatment in the tissue image FA.

After Step S1B, under the control performed by control unit 409, the image processing unit 406 superimposes the treatment target range ArT on the tissue image FA as illustrated in FIG. 9 (Step S1C). Then, the control unit 409 records the data of the tissue image FA, which has the treatment target range ArT superimposed thereon, in the recording unit 408.

As a result of performing the operations from Step S1A to Step S1C, the pre-heat-treatment control method reaches completion.

Control Method Performed when Heat Treatment is Carried Out

Given below is the explanation of the control method implemented by the control device 4 at the time when the heat treatment device 5 performs heat treatment on the body tissue.

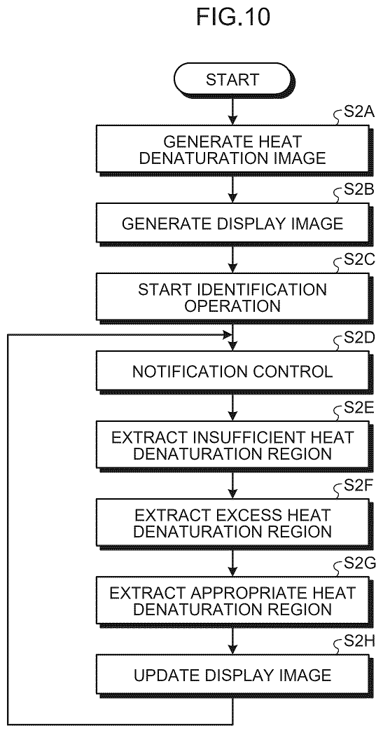

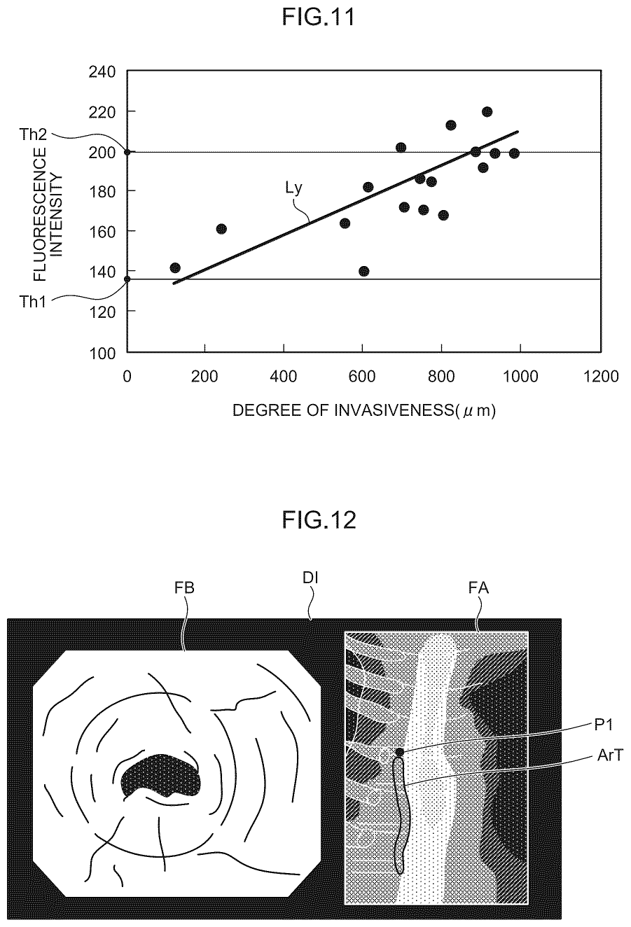

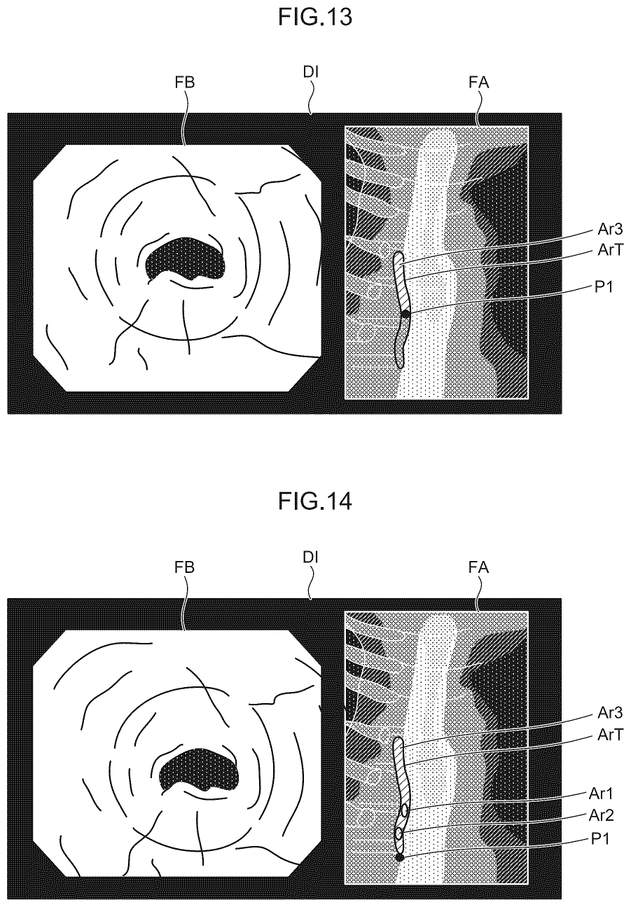

FIG. 10 is a flowchart for explaining the control method performed when heat treatment is carried out. FIGS. 11 to 15 are diagrams for explaining the control method performed when heat treatment is carried out. More particularly, FIG. 11 is a diagram illustrating the correlation (a straight line Ly) between the fluorescence intensity of the autofluorescence of the advanced glycation end product in the body tissue and the degree of invasiveness (the depth and the region) into the body tissue as a result of performing heat treatment. In FIG. 11, the vertical axis represents the fluorescence intensity, and the horizontal axis represents the degree of invasiveness into the body tissue as a result of performing heat treatment. FIG. 12 is a diagram that corresponds to FIGS. 8 and 9 and that illustrates a display image DI generated at Step S2B. FIGS. 13 to 15 are diagrams that correspond to FIG. 12 and that explain the operations from Step S2D to Step S2H.

Meanwhile, the endoscope system 1 is already set to be in the following state.

The insertion portion 21 has been inserted from the mouth into the esophagus of the subject, and accordingly the region inside the esophagus represents the observation region of the endoscope system 1. Moreover, the heat treatment device 5 has been inserted from the insertion opening 222 into the esophagus via the treatment tool channel provided inside the insertion portion 21, and it is possible to perform heat treatment. Moreover, it is assumed that, according to an operation of “switching the observation mode of the endoscope system 1 to the specific observation mode” as performed by the user such as an operator using the operating members 223, the observation mode is switched to the specific observation mode.

Firstly, under the control performed by the control unit 409, the image processing unit 406 generates a heat denaturation image FB (see FIGS. 12 to 15) that enables identification of heat denaturation regions Ar1 to Ar3 in the body tissue (Step S2A). Herein, the heat denaturation region Ar1 represents an insufficient heat denaturation region in which the heat denaturation is not sufficient. The heat denaturation region Ar2 represents an excess heat denaturation region in which the heat denaturation is in excess. The heat denaturation region Ar3 represents an appropriate heat denaturation region in which the heat denaturation is appropriate. In FIGS. 13 to 15, for explanatory convenience, the appropriate heat denaturation region Ar3 is not illustrated in the heat denaturation image FB.

More particularly, in the specific observation mode, alternate switching between the fluorescence observation mode and the normal observation mode is performed, so that a fluorescence image and an observation image (white light image) are generated in a time-sharing manner. Then, at Step S2A, the image processing unit 406 performs a superimposition operation in which the fluorescence image and the observation image (white light image), which are generated at the substantially same timing, are superimposed and the heat denaturation image FB is generated.

Examples of the superimposition operation performed by the image processing unit 406 include a first superimposition operation and a second superimposition operation explained below.

During the first superimposition operation, in an observation image (white light image), the regions having identical pixel positions to the heat denaturation regions Ar1 to Ar3 in a fluorescence image are substituted with images of those heat denaturation regions Ar1 to Ar3.

During the second superimposition operation, according to the fluorescence intensity of each pixel position in the heat denaturation regions Ar1 to Ar3 in a fluorescence image, a change is made in the color brightness that represents the fluorescence assigned to each pixel in the regions indicating the identical pixel positions to the heat denaturation regions Ar1 to Ar3 in an observation image (white light image) (thus, the second superimposition operation represents, what is called, alpha blending).

After Step S2A, the image processing unit 406 reads the data of the tissue image FA from the recording unit 408 at Step S1C and, as illustrated in FIG. 12, generates a display image DI in which the tissue image FA is placed side-by-side with the heat denaturation image FB generated at Step S2A (Step S2B).

After Step S2B, the control unit 409 starts an identification operation for identifying the pixel-by-pixel correspondence relationship between the tissue image FA and the heat denaturation image FB (Step S2C).

More particularly, at Step S2C, based on the magnetism that is coming from the sensor unit 209 and that is received by the receiving unit 411, the control unit 409 estimates the shape of the insertion portion 21 and calculates position information, which indicates the three-dimensional coordinates of the position of the front end of the insertion portion 21 (i.e., the position of the front end portion 24), and calculates direction information, which indicates the field of view of the insertion portion 21. The three-dimensional coordinates represent the coordinates in the same coordinate system as the three-dimensional coordinates assigned to each pixel in the tissue image FA. Moreover, based on the calculated position information and the calculated direction information as well as based on depth map information included in the image data used in generating the heat denaturation image FB, the control unit 409 calculates, for each pixel of the heat denaturation image FB, three-dimensional coordinates of the corresponding position of the observation target that corresponds to the concerned pixel position. Then, the control unit 409 starts the identification operation that includes comparing the three-dimensional coordinates assigned to each pixel of the tissue image FA with the three-dimensional coordinates calculated for each pixel of the heat denaturation image FB, and identifying the pixel-by-pixel correspondence relationship between the tissue image FA and the heat denaturation image FB. Moreover, based on the result of the identification operation and under the control performed by the control unit 409, as illustrated in FIG. 12, the image processing unit 406 superimposes, on the tissue image FA that is displayed in the display image DI, a position P1 in the tissue image FA that represents the subject of the heat denaturation image FB and that corresponds to the position of the region being observed using the endoscope 2.

After Step S2C, the control unit 409 performs notification control (Step S2D).

More particularly, at Step S2D, the control unit 409 controls the operation of the image processing unit 406 and displays the display image DI, which is illustrated in FIG. 12, in the display device 3. Thus, the display device 3 is equivalent to a notification unit.

Meanwhile, as illustrated in FIG. 11, the fluorescence intensity of the autofluorescence of the advanced glycation end product in the body tissue has a correlation with the degree of invasiveness (the height of heat denaturation) into the body tissue as a result of performing heat treatment. More particularly, as illustrated by the straight line Ly in FIG. 11, greater the height of heat denaturation (i.e., greater the degree of invasiveness into the body tissue as a result of performing heat treatment), the greater becomes the fluorescence intensity.

If the insufficient heat denaturation region Ar1 or the excess heat denaturation region Ar2 can be recognized, the user such as an operator becomes able to take the countermeasure and reduce the risk of postoperative recurrence or perforation. In that regard, in the present embodiment, the operations from Step S2E to Step S2H are performed as explained below, so as to make the user, such as an operator, recognize the insufficient heat denaturation region Ar1 or the excess heat denaturation region Ar2 in the treatment target range ArT.

More particularly, from among all of the pixels of the fluorescence image used in generating the heat denaturation image FB, the image processing unit 406 extracts, as the insufficient heat denaturation region Ar1, the region that includes the pixels having the fluorescence intensity equal to or lower than a first fluorescence intensity Th1 (see FIG. 11) (Step S2E).

Moreover, from among all of the pixels of the fluorescence image used in generating the heat denaturation image FB, the image processing unit 406 extracts, as the excess heat denaturation region Ar2, the region that includes the pixels having the fluorescence intensity equal to or higher than a second fluorescence intensity Th2 (see FIG. 11) which is greater than the first fluorescence intensity Th1 (Step S2F).

Furthermore from among all of the pixels of the fluorescence image used in generating the heat denaturation image FB, the image processing unit 406 extracts, as the appropriate heat denaturation region Ar3, the region that includes the pixels having the fluorescence intensity higher than the first fluorescence intensity Th1 and smaller than the second fluorescence intensity Th2 (Step S2G).

The operations from Step S2E to Step S2G either can be performed in that particular order, or can be performed in any other order, or can be performed in parallel in a substantially simultaneous manner.

Examples of the fluorescence intensity used in the operations from Step S2E to Step S2G include the output value of the G pixels in the imaging device 204; at least the g value from among the pixel values (r, g, b) of each pixel after color reconstruction is performed on the image data obtained from the imaging device 204; and the luminance value corresponding to the Y signal (luminance signal).

Then, the image processing unit 406 updates the display image DI (Step S2H). Subsequently, the system control returns to Step S2D.

More particularly, at Step S2H, under the control of the control unit 409, the image processing unit 406 performs the following operation based on the result of the identification operation.

That is, on the tissue image FA that is displayed in the display image DI, the image processing unit 406 superimposes the insufficient heat denaturation region Ar1 in the tissue image FA corresponding to the insufficient heat denaturation region Ar1 in the fluorescence image extracted at Step S2E; superimposes the excess heat denaturation region Ar2 in the tissue image FA corresponding to the excess heat denaturation region Ar2 in the fluorescence image extracted at Step S2F; and superimposes the appropriate heat denaturation region Ar3 in the tissue image FA corresponding to the appropriate heat denaturation region Ar3 in the fluorescence image extracted at Step S2G. Herein, the insufficient heat denaturation region Ar1, the excess heat denaturation region Ar2, and the appropriate heat denaturation region Ar3 are equivalent to heat denaturation information.

As a result of repeatedly performing the operations from Step S2D to Step S2H, the display image DI that is generated by the image processing unit 406 gets modified as explained below.

Firstly, in the state in which the heat treatment is yet to be performed by the heat treatment device 5, the image processing unit 406 generates the display image DI illustrated in FIG. 12.

More particularly, in the display image DI, since the heat treatment is not yet performed, as determined from the position P1, the insufficient heat denaturation region Ar1, the excess heat denaturation region Ar2, and the appropriate heat denaturation region Ar3 are not present in the treatment target range ArT.

When the heat treatment device 5 goes on performing heat treatment, the image processing unit 406 generates the display image DI illustrated in FIG. 13.

More particularly, in the display image DI, as determined from the position P1, of the treatment target range ArT illustrated in FIG. 13, since the heat treatment is performed downward from the topmost portion, at least either the insufficient heat denaturation region Ar1, or the excess heat denaturation region Ar2, or the appropriate heat denaturation region Ar3 is present midway through the treatment target range ArT. In the example illustrated in FIG. 13, only the appropriate heat denaturation region Ar3 is present midway through the treatment target range ArT.

Then, in the state in which the heat treatment device 5 has completed the heat treatment, the image processing unit 406 generates the display image DI illustrated in FIG. 14.

More particularly, in the display image DI, as determined from the position P1, of the treatment target range ArT illustrated in FIG. 14, since the heat treatment is complete till the bottommost portion, at least either the insufficient heat denaturation region Ar1, or the excess heat denaturation region Ar2, or the appropriate heat denaturation region Ar3 is present in the entire treatment target range ArT. In the example illustrated in FIG. 14, the insufficient heat denaturation region Ar1, the excess heat denaturation region Ar2, and the appropriate heat denaturation region Ar3 are present in the entire treatment target range ArT. In FIG. 14, the insufficient heat denaturation region Ar1 is expressed as a hollow circle, the excess heat denaturation region Ar2 is expressed as a dotted circle, and the appropriate heat denaturation region Ar3 is expressed using oblique lines.

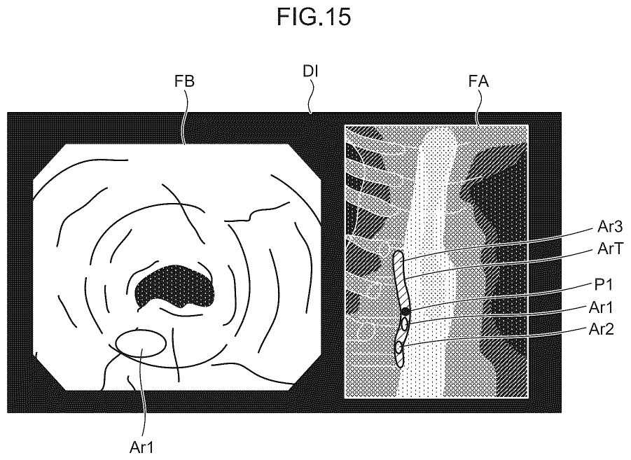

Meanwhile, for example, after the heat treatment is complete, while checking the position P1 in the tissue image FA in the display image D1 that is displayed in the display device 3, if the user such as an operator reverts the front end of the insertion portion 21 to the position corresponding to the insufficient heat denaturation region Ar1, it becomes possible to confirm the insufficient heat denaturation region Ar1 using the heat denaturation image FB as illustrated in FIG. 15.

According to the present embodiment described above, the following effects are achieved.

The control device 4 according to the present embodiment obtains the tissue image FA and the range information indicating the treatment target range ArT. Moreover, based on the fluorescence image, the control device 4 determines the state of the heat denaturation caused by the heat treatment. Then, the control device 4 causes the notification unit to notify about the heat denaturation information indicating the state of heat denaturation in the treatment target range ArT. In the present embodiment, the control device 4 displays, in the display device 3 representing the notification unit, a superimposed image that is obtained by superimposing the heat denaturation information on the tissue image FA. Hence, the control device 4 according to the present embodiment enables the user, such as an operator, to determine whether or not the heat treatment could be appropriately performed in the entire treatment target range Art which is a wide range, and accordingly convenience can be improved.

Moreover, at the time of determining the state of heat denaturation caused by the heat treatment, based on the fluorescence intensity for each pixel in the fluorescence image, the control device 4 according to the present embodiment extracts the insufficient heat denaturation region Ar1, the excess heat denaturation region Ar2, and the appropriate heat denaturation region Ar3.

For that reason, the state of heat denaturation caused by the heat treatment can be determined with ease and in an accurate manner.

### OTHER EMBODIMENTS

Till now, the description was given about the embodiment. However, the disclosure is not limited by the embodiment described above.

In the embodiment described above, the medical device is installed in an endoscope system that is used in peroral endoscopic myotomy (POEM). However, that is not the only possible case. Alternatively, the medical device can be installed in, for example, an endoscope system that is used in inferior turbinate mucosal cautery performed to treat allergic rhinitis or an endoscope system that is used in endometrioma cautery performed to treat endometriosis.

In the embodiment described above, the medical device is installed in an endoscope system including a flexible endoscope. However, that is not the only possible case. Alternatively, the medical device can be installed in an endoscope system including a rigid endoscope or an endoscope system including a surgical robot.

In the embodiment described above, the display device 3 is used as the notification unit. However, that is not the only possible case. Alternatively, as the notification unit, it is possible to use a configuration in which notification is given by displaying an image, or it is possible to use a speaker that gives notification using sounds.

In the embodiment described above, the insufficient heat denaturation region Ar1, the excess heat denaturation region Ar2, and the appropriate heat denaturation region Ar3 are superimposed on the tissue image FA in which the treatment target range ArT is captured; and accordingly the heat denaturation information is displayed that indicates the state of heat denaturation in the treatment target range ArT. However, that is not the only possible case.

Alternatively, for example, only the heat denaturation image FB is displayed without displaying the tissue image FA. Moreover, it is possible to use a configuration in which, when heat treatment of the treatment target range ArT is complete, the heat denaturation information indicating that the heat treatment of the treatment target range ArT has been appropriately completed is notified (using a display of images or characters, or using sounds), or the heat denaturation information indicating that the insufficient heat denaturation region Ar1 or the excess heat denaturation region Ar2 is present in the treatment target range ArT is notified (using a display of images or characters, or using sounds).

In the embodiment described above, as a tissue image, the tissue image FA is used that is a tomographic image taken by a tomographic device. However, that is not the only possible case.

Alternatively, for example, using the SLAM technology (SLAM stands for Simultaneous Localization and Mapping), endoscope images (observation images (white light images)) taken by the endoscope 2 can be linked and the resultant image (hereinafter, referred to as a linked image) can be used as the tissue image.

Meanwhile, the tissue image can either be a two-dimensional image or be a three-dimensional image.

In the embodiment described above, as a result of using a configuration having the TOF sensor included in the imaging device 204, the sensor unit 209, and the receiving unit 411; the control device 4 calculates the three-dimensional coordinates of the position of the front end of the insertion portion 21 (i.e., the position of the front end portion 24). However, that is not the only possible case.

Alternatively, for example, the TOF sensor, the sensor unit 209, and the receiving unit 411 can be omitted, and the control device 4 can be configured to calculate the three-dimensional coordinates of the front end of the insertion portion 21 using the linked image mentioned above.

In the embodiment described above, the control device 4 performs image recognition using the learning model recorded in the learning model recording unit 408b, and obtains (extracts) the range information indicating the treatment target range ArT in the tissue image FA. However, that is not the only possible case.

Alternatively, for example, while displaying the tissue image FA in the display device 3, the control device 4 cab obtain, as the treatment target range ArT, the range selected in the tissue image FA by the user such as an operator by operating the input unit 407.

According to the medical device, the endoscope system, the control method, and the computer program product according to the disclosure, convenience can be improved.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical device comprising a processor configured to:
obtain a tissue image including a target for a heat treatment,
obtain a fluorescence image that is taken by an imaging sensor,
identify a correspondence relationship between the tissue image and the fluorescence image,
obtain relationship information on a correlation between a fluorescence intensity in the fluorescence image and a degree of thermal invasiveness,
identify an insufficient heat denaturation region based on the fluorescence image and the relationship information,
identify an excess heat denaturation region based on the fluorescence image and the relationship information,
identify an appropriate heat denaturation region based on the fluorescence image and the relationship information,
add heat denaturation information including the insufficient heat denaturation region, the excess heat denaturation region, and the appropriate heat denaturation region, to the tissue image based on the correspondence relationship between the tissue image and fluorescence image, and
superimpose the insufficient heat denaturation region, the excess heat denaturation region, and the appropriate heat denaturation region on the tissue image such that the insufficient heat denaturation region, the excess heat denaturation region, and the appropriate heat denaturation region are identifiable from each other, and display resultant image in a display.

2. The medical device according to claim 1, wherein the fluorescence image is an image that is obtained by capturing fluorescence generated from an advanced glycation end product that is generated due to the heat treatment performed on a body tissue.

3. The medical device according to claim 1, wherein, from among all pixels of the fluorescence image, the processor is configured to identify, as the insufficient heat denaturation region in which heat denaturation caused by the heat treatment is not sufficient, a region that includes pixels having a fluorescence intensity equal to or lower than a first fluorescence intensity.

4. The medical image according to claim 1, wherein, from among all pixels of the fluorescence image, the processor is configured to identify, as the excess heat denaturation region in which heat denaturation caused by the heat treatment is in excess, a region that includes pixels having a fluorescence intensity equal to or higher than a second fluorescence intensity which is higher than the first fluorescence intensity.

5. The medical device according to claim 1, wherein the tissue image is either a tomographic image taken by a tomographic device or an ultrasonic image generated by an ultrasonic observation device.

6. The medical device according to claim 1, wherein the tissue image is generated by linking endoscope images which are taken by an endoscope.

7. The medical image according to claim 1, wherein
the fluorescence image is an endoscope image taken by an endoscope that includes an insertion portion configured to be inserted inside a subject, and
the processor is configured to
calculate three-dimensional coordinates of the subject in the fluorescence image based on position information indicating a position of a front end of the insertion portion, based on direction information indicating a field of view of the front end, and based on the fluorescence image, and
identify a pixel-by-pixel correspondence relationship between the tissue image and the fluorescence image based on the three-dimensional coordinates.

8. An endoscope system comprising:
a light source configured to emit an excitation light;
an endoscope configured to output a taken image which is taken by an imaging sensor; and
a medical device comprising a processor configured to process the taken image, the processor being configured to
obtain a tissue image including a target for a heat treatment,
obtain a fluorescence image that is taken by the imaging sensor,
identify a correspondence relationship between the tissue image and the fluorescence image,
obtain relationship information on a correlation between a fluorescence intensity in the fluorescence image and a degree of thermal invasiveness,
identify an insufficient heat denaturation region based on the fluorescence image and the relationship information,
identify an excess heat denaturation region based on the fluorescence image and the relationship information,
identify an appropriate heat denaturation region based on the fluorescence image and the relationship information,
add heat denaturation information including the insufficient heat denaturation region, the excess heat denaturation region, and the appropriate heat denaturation region, to the tissue image based on the correspondence relationship between the tissue image and fluorescence image, and
superimpose the insufficient heat denaturation region, the excess heat denaturation region, and the appropriate heat denaturation region on the tissue image such that the insufficient heat denaturation region, the excess heat denaturation region, and the appropriate heat denaturation region are identifiable from each other, and display resultant image in a display.

9. A control method implemented in a medical device, comprising:
obtaining a tissue image including a target for a heat treatment,
obtaining a fluorescence image that is taken by an imaging sensor,
identifying a correspondence relationship between the tissue image and the fluorescence image,
obtaining relationship information on a correlation between a fluorescence intensity in the fluorescence image and a degree of thermal invasiveness,
identifying an insufficient heat denaturation region based on the fluorescence image and the relationship information,
identifying an excess heat denaturation region based on the fluorescence image and the relationship information,
identifying an appropriate heat denaturation region based on the fluorescence image and the relationship information,
adding heat denaturation information including the insufficient heat denaturation region, the excess heat denaturation region, and the appropriate heat denaturation region, to the tissue image based on the correspondence relationship between the tissue image and fluorescence image, and
superimposing the insufficient heat denaturation region, the excess heat denaturation region, and the appropriate heat denaturation region on the tissue image such that the insufficient heat denaturation region, the excess heat denaturation region, and the appropriate heat denaturation region are identifiable from each other, and displaying resultant image in a display.

10. A non-transitory computer-readable recording medium with an executable program stored thereon, the program causing a medical device to execute:
obtaining a tissue image including a target for a heat treatment,
obtaining a fluorescence image that is taken by an imaging sensor,
identifying a correspondence relationship between the tissue image and the fluorescence image,
obtaining relationship information on a correlation between a fluorescence intensity in the fluorescence image and a degree of thermal invasiveness,
identifying an insufficient heat denaturation region based on the fluorescence image and the relationship information,
identifying an excess heat denaturation region based on the fluorescence image and the relationship information,
identifying an appropriate heat denaturation region based on the fluorescence image and the relationship information,
adding heat denaturation information including the insufficient heat denaturation region, the excess heat denaturation region, and the appropriate heat denaturation region, to the tissue image based on the correspondence relationship between the tissue image and fluorescence image, and
superimposing the insufficient heat denaturation region, the excess heat denaturation region, and the appropriate heat denaturation region on the tissue image such that the insufficient heat denaturation region, the excess heat denaturation region, and the appropriate heat denaturation region are identifiable from each other, and displaying resultant image in a display.
